(19) United States
(12) Patent Application Publication
(10) Pub. No.: US 2023/0277710 A1
(43) Pub. Date: Sep. 7, 2023

(54) SYSTEM AND DEVICE FOR SANITIZING THE AIR

(71) Applicant: The Procter & Gamble Company, Cincinnati, OH (US)

(72) Inventors: Dana Paul GRUENBACHER, Fairfield, OH (US); Julie Ann O'NEIL, Dillsboro, IN (US); Champak DAS, Gainesville, FL (US); Thomas Paul HARTRANFT, Clearwater, FL (US); Michael Robert EIDELL, Madeira, OH (US)

(21) Appl. No.: 18/115,793

(22) Filed: Mar. 1, 2023

Related U.S. Application Data
(60) Provisional application No. 63/315,104, filed on Mar. 1, 2022.

(51) Int. Cl. A61L 9/03; F24F 8/10; F24F 8/24

(52) U.S. Cl. CPC A61L 9/032; F24F 8/10; F24F 8/24; A61L 9/037; A61L 2209/111; A61L 2209/14; A61L 2209/21; A61L 2209/135

(57) ABSTRACT

An air sanitizing system is provided. The air sanitizing system includes a dispensing device configured to dispense a sanitizing composition into liquid particles and vapor. The air sanitizing system also includes a fan that is configured to disperse the vapor of sanitizing composition into the air. The air sanitizing system includes a filtering device configured to remove at least a portion of the liquid particles.

### FIELD

The present disclosure is directed to a system and device for sanitizing the air, and, more specifically, is directed to a system and device for sanitizing the air including a dispensing device and a filtering device to remove dispensed liquid particles of sanitizing composition from the air.

### BACKGROUND

Sanitizing compositions, such as compositions containing triethylene glycol, are known for sanitizing the air. Devices that emit such sanitizing compositions into the air are also known. However, such devices may emit both vapor and liquid particles of the sanitizing composition into the air. Liquid particles of the sanitization composition in the air can cause a haze in the air that is undesirable by some consumers. As such, a system, device, and method for sanitizing the air which reduces the amount of haze created, can remove the haze from the air or does not create haze when sanitizing is desired.

### SUMMARY

“Combinations:”
A. An air sanitizing system comprising:
   a dispensing device configured to dispense a sanitizing composition in the form of liquid particles and vapor into the air;
   a fan;
   a filtering device configured to remove at least a portion of the liquid particles.
B. The system of Paragraph A, wherein the dispensing device comprises a pump with an evaporative surface.
C. The system of any of Paragraphs A and B, wherein the dispensing device comprises a heater.
D. The system of any of Paragraphs A through C, wherein the dispensing device comprises the filtering device.
E. The system of any of Paragraphs A through C, wherein the dispensing device and the filtering device are discrete parts of the system.
F. The system of any of Paragraphs A through E, wherein the filtering device comprises at least one of a circular vortex, an elbow, or a conical-shaped mesh configured to collect the liquid particles on a surface of the dispensing device.
G. The system of any of Paragraphs A through F, wherein the filtering device comprises a first filter, wherein the dispensing device comprises the filtering device, and wherein the filtering device comprises a second filter separate from the first filter.
H. The system of any of Paragraphs A through I, wherein the sanitizing composition comprises a glycol ether.
I. The system of Paragraph H, wherein the glycol ether comprises triethylene glycol.
J. The system of Paragraph I, wherein the sanitizing composition comprises at least 90 wt. % triethylene glycol, by total weight of the sanitizing composition.
K. The system of any of Paragraphs A through J further comprising a sensor configured to measure the level of liquid particles or vapor in the air.
L. The system of any of Paragraphs A through K further comprising a feedback loop.
M. The system of any of Paragraphs A through L further comprising a sensor configured to measure the level of one or more airborne microorganisms in the air.
N. The system of any of Paragraphs A through M, wherein the dispensing device comprises a microfluidic die.
O. The system of any of Paragraphs A through N, wherein the dispensing device comprises a wick.
P. An air sanitizing system comprising:
   a dispensing device comprising a filtering device comprising a first filter, wherein the dispensing device is configured to dispense a sanitizing composition;
   a fan; and
   a second filter separate from the first filter.
Q. The system of Paragraph P, wherein the dispensing device comprises a pump, an evaporative surface for receiving a sanitizing composition from the pump, and a heater disposed adjacent to the evaporative surface for evaporating the sanitizing composition.
R. The system of Paragraph Q, wherein the sanitizing composition comprises a glycol ether.

### BRIEF DESCRIPTION OF THE DRAWINGS

FIG. 1 is a perspective view of an air sanitization system that includes a dispensing device and a filtering device.

FIG. 2 is a cross-sectional view of the dispensing device of FIG. 1.

FIG. 3 is a perspective view of a dispensing device for filtering liquid particles of a sanitizing composition and dispensing vapor of the sanitizing composition.

FIG. 4 is a cross-sectional view of the dispensing device of FIG. 3.

FIG. 5 is a perspective view of a dispensing device for filtering liquid particles of a sanitizing composition and dispensing vapor of the sanitizing composition.

FIG. 6 is a cross-sectional view of the dispensing device of FIG. 5.

FIG. 7 is a side elevation view of a dispensing device for dispensing a sanitizing composition that is intended to be plugged into an electrical outlet in a wall.

FIG. 8 is a perspective view of a dispensing device that includes a microfluidic die for dispensing a sanitizing composition.

FIG. 9 is a perspective view of a cartridge for the dispensing device of FIG. 8.

FIG. 10 is a graph showing the concentration of triethylene glycol in the air versus time showing the effect of heating as disclosed herein on the same.

FIG. 11 is a graph showing the concentration of triethylene glycol in the air versus time for a heated and an unheated variant showing the effect of filtration on the same.

FIG. 12 is a graph showing the concentration of triethylene glycol in the air versus time for a heated and an unheated variant showing the effect of another filtration media on the same.

### DETAILED DESCRIPTION

Sanitization systems of the present disclosure can disperse sanitizing compound into the air to kill or deactivate microorganisms in the air. Additionally, the sanitization systems of the present disclosure remove at least a portion of the liquid particles of sanitizing composition in the air either by reducing the amount of liquid particles being dispensed and/or by filtering the liquid particles from the air. This can result in less haze being generated as explained further herein.

The systems and methods of sanitizing the air, in accordance with the present disclosure, include a dispensing device for dispensing the sanitizing composition into the air. The dispensing device of the present disclosure has a dispensing device housing and can generate and dispense vapor as well as liquid particles of sanitizing composition. As noted, large liquid particles can cause haze. So, the systems and methods of the present disclosure also can reduce the number of liquid particles in the air. Without wishing to be bound by theory, it is believed that the sanitizing composition vapor provides a much better kill/deactivation of airborne microorganisms than do liquid particles. Preferably the systems and methods of the present disclosure involve the dispersion of primarily vapor into the air. The sanitizing composition may be dispensed into the air through a variety of means, including heat, air flow, atomization, and other means of putting vapor or small particles into the air to enable evaporation.

The sanitizing composition may include one or more active compounds that kill or deactivate microorganisms in the air. The sanitizing composition may be in liquid form. The active compound may be selected from the group consisting of: triethylene glycol (TEG), polyethylene glycol (PEG), dipropylene glycol (DPG), propylene glycol (PG), thymol, essential perfume oils, short-chain alcohols such as ethanol, bioflavonoids, hypochlorous acid, hydrogen peroxide, and other know biocides. The sanitizing composition may be comprised of 100% active compound, or the sanitizing composition may include a solvent. One or more solvents such as water ethanol, low-chain alcohols, and the like may be used in the sanitizing composition. The active compound may be present at a level of about 50 wt. % to about 100 wt. %, more preferably at least 75 wt. %, even more preferably at least 90 wt. % active compound, even more preferably at least 95 wt. % or most preferably 100 wt. % active compound, based on the total weight of the sanitizing composition, specifically including all values within these ranges and any ranges created thereby.

Airborne microorganisms include bacteria and viruses. Exemplary airborne viruses include rhinoviruses, influenza viruses (e.g. type A, type B, H1N1), varicella viruses, measles virus, mumps virus, hantavirus, viral meningitis, COVID. Exemplary airborne bacteria include gram-positive bacteria such as staphylococci (Staphylococcus aureus, S. epidermidis,) streptococci (Streptococcus pyogenes, S. pneumoniae, etc.), enterococci, and Clostridium difficile, C. perfringens, Listeria monocytogenes. Exemplary gram-negative bacteria include Pseudomonas, klebsiella, proteus, salmonella, providencia, escherichia, morganella, aeromonas, and citrobacter. occasionally, gram-positive organisms (e.g., streptococcus, corynebacteria).

As noted, the dispensing device of the present disclosure can disperse both sanitizing composition vapor and liquid particles into the air. There are a myriad of ways to create sanitizing vapor. As an example. the dispensing device of the present disclosure can create and dispense very small liquid particles that are able to evaporate quickly (in less than 5 mins and preferably less than 1 min). These very small liquid particles may be less than 5 μm and more preferably less than 1 μm. A sprayer or atomization device that can generate very small liquid particles would include thermal Ink-jet systems, micro apertured sprayers, mesh nebulizers, jet nebulizers, ultrasonic nebulizers, high flow-through pump sprayer, and the like. In other example, sanitizing vapor may be created via ultrasonic or piezo vibration, or via venturi which aerosolize the sanitizing composition into vapor, and/or the sanitizing composition can be evaporated via heat, airflow, or a combination thereof.

In order to accelerate evaporation, the surface area of the sanitizing composition may be increased by spraying or atomizing the sanitizing composition into the air or onto an evaporative surface that is either heated or unheated. Evaporation may be accelerated by using a heated surface with a porous heat conducting material (metal mesh, foam, porous metal) that helped the sanitizing composition spread-out while also increasing the surface area that the sanitizing composition was in contact with the heated surface.

Where heat is desired as the mechanism for vapor creation, the sanitizing composition can be heated in a variety of ways, including but not limited to, electrical resistance heaters such as a cartridge heater or thin film printed heaters, or any heater that uses resistors to create heat energy. Heating can also be accomplished through infrared, ultrasonics, microwaves and other similar approaches. When heating the sanitizing composition, the temperature needed for evaporation will depend on the boiling point of the sanitizing composition. It may be desirable to heat the sanitizing composition to a temperature below the boiling point of the sanitizing composition. Heating the sanitizing composition at or above the boiling point of the sanitizing composition may cause degradation of the sanitizing composition over time. In a non-limiting example where the sanitizing composition comprises 100 wt. % TEG, which has a boiling point of 285° C., the sanitizing composition may be heated to a temperature of about 225-255° C. to avoid degradation of the TEG.

As discussed above, the sanitizing composition may be evaporated from an evaporative surface. The evaporative surface may be configured as a porous or semi-porous substrate, a bowl or plate, including a plastic, glass, or metal bowl or plate, and combinations thereof. The sanitizing composition may be delivered to the evaporative surface with a pump, such as a micro-pump, that is able to control the amount of sanitizing composition that is being evaporated. Suitable micropumps can include peristaltic pumps, positive displacement pumps, piezo pumps, diaphragm pumps among others. It may be desirable to control the sanitizing composition rate delivered to the evaporative surface to a rate of 0.05 milliliters per minute (mL/min) to 10 mL/min. However, the rate and amount of evaporation can be selected to the evaporative levels desired for the room size or treatment area for the application.

The systems/devices and method of sanitizing the air can utilize a fan to assist in evaporation of the sanitizing composition. A fan can also help circulate the vapor of sanitizing composition from a dispensing device and into the air. Any suitable fan may be utilized. Preferably, the fan can move a sufficient amount of air to create at least 3 air exchanges per hour or higher. For example, preferably the fan provides a sufficient output such that between about 3 to about 8 air exchanges per hour occur, more preferably from between about 4 to about 8 air exchanges per hour, or most preferably about 5 to about 8 air exchanges per hour occur, specifically reciting all values within these ranges and any ranges created thereby. Higher air exchanges will circulate the sanitizing vapor through the room more quickly and can achieve the 3-log (99.9%) reduction more quickly as well. Additionally, higher air exchanges rates can remove particles from the air more quickly as well which can result in a reduced amount of haze.

It is worth noting that the system and/or device of the present disclosure may include more than one fan or a single fan may be utilized. For the circulation of sanitizing composition from the device, any suitable fan capacity may be utilized. For example, circulation of the sanitizing composition can be accommodated by a fan providing from between 10 cubic feet per minute (cfm) to about 200 cfm, more preferably from about 20 cfm to about 150 cfm, even more preferably from about 22 cfm to about 100 cfm, even more preferably from about 25 cfm to about 75 cfm, or even more preferably from about 30 cfm to about 50 cfm, specifically reciting all values within these ranges and any ranges created thereby. It is believed that at too low of a cfm, local saturation can increase which can lead to liquid particles. Further, it is believed that too high of an airflow can create problems with temperature control particularly for the evaporative surface and/or inlet airstream and can similarly lead to increased liquid particle formation. Depending on the size of the space to be sanitized and the desired air exchange rate, one or more systems/devices of the present disclosure may be utilized.

As noted herein, for the reduction of haze created by the sanitizing composition in the air, filtering of the air may be utilized. In general, the filtering process utilizes air flow to move air through a filtering device which removes particles from the air flowing therethrough. In order to remove the haze as quickly as possible, a fan can be used in the filtering device that has a capacity which is higher than that utilized for the evaporation of the sanitizing composition. For example, during filtering a separate fan or the same fan (via modification of fan speed) may be utilized for evaporation may provide from about 70 cfm to about 500 cfm, more preferably from about 100 cfm to about 400 cfm, even more preferably from about 125 cfm to about 300 cfm, or most preferably from about 150 cfm to about 200 cfm, specifically reciting all values within these ranges and any ranges created thereby.

Systems with fans which provide less than 3 air exchanges per hour can still provide the 3-log (99.9%) reduction; however, the time to achieve this effect will be longer than systems with fans capable of producing higher air exchange rates. Similarly, the lower air exchange rates can also mean that any liquid particles in the air are present for a longer time period. This can result in the haze being present for a longer period of time.

Where a heater is utilized, the fan may be positioned upstream or downstream of the heater. Care should be taken to ensure that the airflow from the fan is appropriately routed to ensure that the heater is not constantly being cooled by airflow.

It has been observed that a relatively large number of liquid particles may be generated when the saturation level for the air temperature and humidity conditions is exceeded for low vapor pressure sanitizing compounds such as TEG and other glycols. As noted, relatively large liquid particles that can create a visible haze effect in the air. The larger liquid particles can also be slower to evaporate and can lead to wetness on nearby surfaces.

In order to reduce the number of liquid particles of sanitizing composition in the air, and particularly relatively large liquid particles, the liquid particles may be removed from the air while still producing the desired sanitization effect in the air by maintaining the vapor of sanitizing composition in the air. As described hereinafter, measures may be taken to reduce the formation of liquid particles and ensure that a larger amount of vapor is created instead. In addition, or independently thereof, the liquid particles that are formed may be removed from the air via a filtering device as described herein.

Regarding the reduction of the formation of liquid particles, it has been found that lower evaporation temperatures, increased air flow, and/or higher surface area at an evaporative surface can minimize liquid droplet formation of the sanitizing composition in the air. With higher evaporation temperatures, the sanitizing composition may saturate the head space and encourage condensation in the air and/or on surfaces. So, lower temperatures for controlled evaporation can be utilized to ensure the headspace does not get saturated.

Additionally, the inventors have surprisingly found that airflow utilized to disperse the vapor can facilitate the formation of liquid particles. For example, where the air temperature is below that of condensation for the sanitizing composition, liquid particles can be formed. Any suitable method of heating the airflow going into or through the device may be utilized. Some examples include, but are not limited to, resistance heating, infrared heaters, microwave, heat exchangers, and the like.

Where the air going into the device or through the device is heated any suitable temperature may be utilized. However, overall configuration of the dispensing device or system can make a difference as to the appropriate temperatures to utilize. As an example, in order to remove liquid particles from entering the room air, the dispensing device may comprise a filtering device which is attached to or immediately adjacent an outlet of the dispensing device. However, where the filtering device is subjected to higher temperatures of air and where the filtering device retains the captured liquid particles, e.g. media filter, too high a temperature can lead to additional evaporation of the liquid particles captured by the filtering device. And while conceptually beneficial, the increase in sanitizing composition in the air can lead to an oversaturation of the air which can in turn lead to the generation of haze. The inventors have found that the temperature of the air at the outlet of the device can be above room temperature to help reduce the generation of liquid particles. For example, the temperature of the air at the outlet can be greater than 22 degrees C. to less than about 50 degrees C., more preferably from about 25 degrees C. to about 40 degrees C., even more preferably from about 27 degrees C. to about 35 degrees C. or most preferably from about 29 degrees C. to about 32 degrees C., specifically reciting all values within these ranges and any ranges created thereby.

In contrast, where the dispensing device and/or system of the present disclosure does not include a filtering device or where the filtering device is positioned such that the filtering device is not subjected to the elevated air temperature, then the air at the outlet of the dispensing device can be greater than 22 degrees C. to about 65 degrees C., more preferably from about 25 degrees C. to about 65 degrees C., even more preferably from about 30 degrees C. to about 65 degrees C., or most preferably from about 32 degrees C. to about 65 degrees C., specifically reciting all values within these ranges and any ranges created thereby.

It is worth noting that some configurations of the evaporative surface, particularly when heated, may obviate the need for another heater. For example, the evaporative surface may be configured such that it comprises fins or similar heat transfer mechanism to heat the air as it passes adjacent, over, and/or under the evaporative surface. While the use of this type of evaporative surface may negate the need for a separate heater, such configurations may be more difficult to control from a temperature standpoint.

While temperature, air flow, and evaporative surface area may minimize the formation of liquid particles, it has been found that filtering the liquid particles reduces the level of haze in the air even further. Regarding the filtering of the air in the room, any suitable filtering device may be utilized. The filtering device should be capable of removing 1 micron and above particles. Particularly, the particles in the size range of 1-10 micron and larger may tend to float in the air and contribute to haze generation. In contrast, smaller particles have much less mass and do not contribute to haze generation to the extent of the particles greater than 1 micron particles. Of the 1-10 micron sized particles, the 5 micron-10 micron size and greater contribute more to haze than those particles which are less than 5 micron. Similarly, the 2.5 micron-10 micron range contribute more to haze generation than those particles which are less than 2.5 micron. Additionally, particles over 10 microns in size will tend to fall out of the air given their larger size and therefore also not contribute to haze generation to the same extent as the 1-10 micron particles.

Filtering devices of the present disclosure may utilize a variety of techniques to remove particles such as filter media, electrostatics, ionization and other means to remove particles or droplets. Electrostatics, ionization and other approaches can cause particles to be attracted to a collection plate, fibers or other surfaces that can either be cleaned or disposed. There are also techniques to make particles agglomerate in the air, so they are easier to remove or heavy enough to fall down or collect on a surface to remove. Exemplary media filters include a HEPA filter, a pleated filter, non-woven filter, glass fiber filter, foam-based filter, metal mesh filter, or a bag to remove such liquid particles including a HEPA filter and/or a non-woven bag filter. Suitable examples of filter media include any combination of fibers, granules, plates, and structures that can let air pass through while also removing the particles.

The filtering device may either be attached with the dispensing device such that the vapor and liquid particles pass through this filtering device before the vapor are released into the space. The filtering device may also be a separate unit that filters out liquid particles that are dispensed into the air from the dispensing device. For example, the filtering device could be part of building HVAC system or can be a separate device such as a portable room air purifier or the like. The filtering device may include a filter in or attached to the dispensing device and a separate second filter located in the same space but away from the dispensing device. Additional devices for removal of liquid particles from an air stream are described herein.

As noted, the filtering device may be positioned in any suitable location with respect to the dispensing device. For example, the dispensing device may be attached to the filtering device in such a way that the sanitizing composition, including vapor and liquid particles, are dispensed into the air and the attached filtering device is used to filter out the liquid particles that are dispensed into the air. Furthering this example, the filtering device may be positioned at the outlet or inlet of the dispensing device (unitary) or the filtering device may be positioned in any other suitable location which allows for the filtration of particles from the room air, e.g., adjacent the outlet or the inlet of the device. Where filtering of the particles from the room air is not occurring or where a filtering device is not utilized, it is believed that a higher amount of sanitizing composition may be provided to the air. The higher amount of sanitizing composition can help achieve a 3-log (99.9%) reduction of airborne microorganisms faster than when filtering is occurring simultaneously with dispersion. The delay of filtering of the particles is discussed in additional detail herein.

Where the dispensing device comprises the filtering device, the filtering device and/or dispensing device may include a button or switch that allows a user to turn the dispensing device ON or OFF at the user's discretion. The filtering device and/or dispensing device may also be programmed such that the dispensing device turns ON and OFF at certain times or when certain conditions are met (i.e. the room is empty).

If the filtering device is a separate unit, the air flow through the filtering device may be sufficient to clean the room in less than 15 minutes. The filtering device may produce at least two air exchanges per hour, or preferably more than four air exchanges per hour through the filtering device. The filtering device may trap all or substantially all of the liquid particles that were dispensed from the dispensing device.

Regardless of the location of the filtering device, if one is utilized, the filtering device is preferably capable of removing at least 99% of 5 micron and above sized particles in less than or equal to about 20 minutes, more preferably less than or equal to about 15 minutes, even more preferably less than or equal to about 10 minutes or most preferably less than or equal to about 5 minutes, specifically including all values within this range and any ranges created thereby. The filtering device utilized for the systems/dispensing devices of the present disclosure are preferably capable of removing particles being 2.5 microns or greater in size. For example, the filtering device of the systems/devices of the present disclosure may remove at least 70%, more preferably at least 80%, even more preferably at least 90%, or most preferably at least 99% of particles being 2.5 microns or greater in size, specifically including all values within these ranges and any ranges created thereby. Preferably the filtering devices utilized in the systems/dispensing devices of the present disclosure can remove from between 70% and 99% of particles having a size of 2.5 microns or greater in less than 20 minutes, more preferably less than or equal to 15 minutes, even more preferably less than or equal to 10 minutes or most preferably less than or equal to 5 minutes, specifically including all values within these ranges and any ranges created thereby.

The reduction of liquid particles from the air may occur contemporaneously with the operation of the dispensing device of the present disclosure. For example, when the dispensing device is in operation, at time zero when the dispersion of the sanitizing composition begins, the removal of liquid particles can similarly be contemporaneous or with very short lag between dispersion and removal of liquid particles from the air. However, as noted previously, a 3-log reduction (99.9% reduction) in airborne microorganisms can occur much quicker where the systems/dispensing device of the present disclosure do not include the use of a filtering device or when the removal of the liquid particles from the air, particularly where a filtering device is used, is delayed by a period of time. The removal of liquid particles from the air can be delayed from time zero (time of initial dispensing of the sanitizing composition) from between about 2 minutes to about 30 minutes, more preferably from about 3 minutes to about 20 minutes, or even more preferably from about 4 minutes to about 10 minutes, specifically including all values within these ranges and any ranges created thereby.

Air flow, concentration of the sanitizing composition in the air, and dwell time of the sanitizing composition in the air can be varied to control the desired airborne microorganism reduction level. The desired airborne microorganism reduction may be 1-log reduction (90% reduction), 2-log reduction (99% reduction), 3-log reduction (99.9% reduction), or 4-log reduction (99.99%). The airborne microorganism reduction may occur in less than 60 minutes, preferably less than 50 minutes, more preferably less than 40 minutes, even more preferably less than 30 minutes, even more preferably less than 20 minutes or most preferably from between 2 to 10 minutes, specifically including all values within these ranges and any ranges created thereby.

With the method of the present disclosure, particularly where filtering of the liquid particles is delayed as described herein, a 3-log (99.9%) reduction is possible in under 10 minutes after dispersion of the sanitizing composition begins, preferably less than 8 minutes, and more preferably less than 6 minutes, specifically reciting all values within these ranges and any ranges created thereby. For example, the 3-log reduction (99.9% reduction) can occur in a time frame of between about 2 minutes to about 10 minutes after the dispersion of the sanitizing composition begins, more preferably from about 2 minutes to about 8 minutes, or most preferably from about 2 minutes to about 6 minutes, specifically including all values within these ranges and any ranges created thereby.

Where filtration of the liquid particles is concomitant or within a short period of time, e.g., less than 1 minute, from the commencement of sanitizing composition dispersion, then a 3-log reduction (99.9% reduction) can occur in about 10 to about 20 minutes, more preferably from about to about 15 minutes, specifically including all values within these ranges and any ranges created thereby.

As mentioned herein, there are a number of ways to reduce the amount of liquid particles dispersed into the air. Any suitable combination of these ways, e.g., lower evaporation temperatures, increased airflow, higher surface area at an evaporative surface, heating air flowing through the device, and filtering may be utilized in order to reduce the amount of liquid particles dispersed into the air.

The devices and systems of the present disclosure can provide sanitizing composition into the air at a concentration of about 0.1 mg/m³ to about 35 mg/m³, or 0.5 mg/m³ to 10 mg/m³, or 1 mg/m³ to 5 mg/m³, specifically reciting all values within these ranges and any ranges created thereby.

It is worth noting that the system of the present disclosure may comprise a unitary structure or may comprise a plurality of discrete parts. For example, the evaporative surface and fan may be disposed in the dispensing device housing. However, the filtering device and/or a heat source may be separate from the dispensing device housing. In contrast, the evaporative surface, fan, filtering device and/or heat source may be disposed in the dispensing device housing.

The devices/systems of the present disclosure may comprise one or more sensors. Examples of sensors that may be utilized are particle sensors, temperature sensors, humidity sensors, optical sensors, chemistry sensor, VOC sensors, occupancy sensors or the like. These sensors may be utilized to control a variety of functions in the systems/devices of the present disclosure, e.g. fan speed, evaporation temperature, filtration ON/OFF and the like. The sensor(s) may be in signal communication, either directly or indirectly (via a microprocessor) and either wired or wirelessly, with the fan, heater, pump, filtering device or the like. For example, temperature and/or humidity sensors may provide input to, for example, a heater or fan to control the evaporation rate of the sanitizing composition. This can reduce the likelihood of local saturation at the dispensing device. Similarly, a particle sensor that can measure the level of sanitizing composition or active compounds in the sanitizing composition at both a fan air inlet or an air flow exit and ideally can measure the amount of sanitizing composition or active compounds before and after in air so as to enable good control of the level of active compound in the room to maintain the micro reduction level while minimizing haze in the air. For example, the one or more sensors may be in signal communication, directly or indirectly via a microprocessor, with at least one of the fan, the filtering device, the heater or the pump. The one or more sensors may be utilized to control the concentration level of the sanitizing composition that is in the air. Non-limiting examples of sensors that can be used to detect the concentration would include VOC sensors, chemical sensors, and particle sensors or any combinations thereof. The sensors may detect particles between 0.3 μm and 10 μm or larger.

Additionally, or independently thereof, the devices and/or systems of the present disclosure may comprise an evaporation sensor that detects the amount of sanitizing composition that has been evaporated. After a desired amount of sanitizing composition has been evaporated and circulated in the air, the particle sensor may be utilized to determine the amount of particles in the air versus the amount of vapor. If the particle sensor determines that a certain level of particles are in the air, the particle sensor may then provide a signal which causes the filtering device to turn on or may start a timer to delay the start of filtration of the liquid particles from the air.

The sensor(s) may be in signal communication, either wired or wirelessly, to a microprocessor which is in signal communication to the fan, if present, and/or to the evaporative device. As an example, based on the temperature and humidity of the room, the sensor(s) may establish a saturation level of the sanitizing composition in the air, e.g., about 5 mg/m³. Once the devices/systems of the present disclosure approach this saturation level, the sensor(s) may reduce the temperature of the evaporative surface to slow the rate of evaporation. As another example, sensor(s) may be in signal communication, either wired or wirelessly, to the microprocessor which is in signal communication with a filtering device. The sensor(s) and/or microprocessor may comprise a clock circuit which turns on the fan after a certain period of time as expressed previously. The systems of the present disclosure may be equipped with a timer which delays the start of filtration by a desired amount (as described herein) to allow a longer dwell time of sanitizing composition in the air.

The system can also use occupancy sensors to recognize when people are present and for how long to automatically adjust the concentration of the sanitizing composition in the air based on occupancy time or desired action (e.g. disinfect/sanitize air in room in between guests in a hotel room vs. maintain desired level with people present for several hours). In such configurations, the occupancy sensor may be in signal communication, either wired or wirelessly, with the microprocessor which is in signal communication with the fan, evaporator, and/or filtering device.

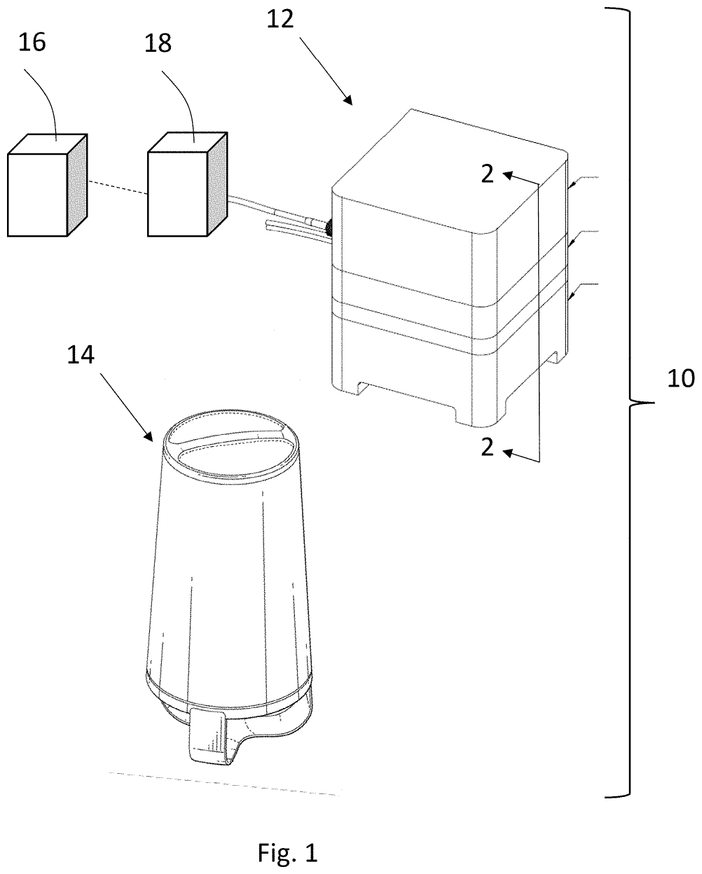

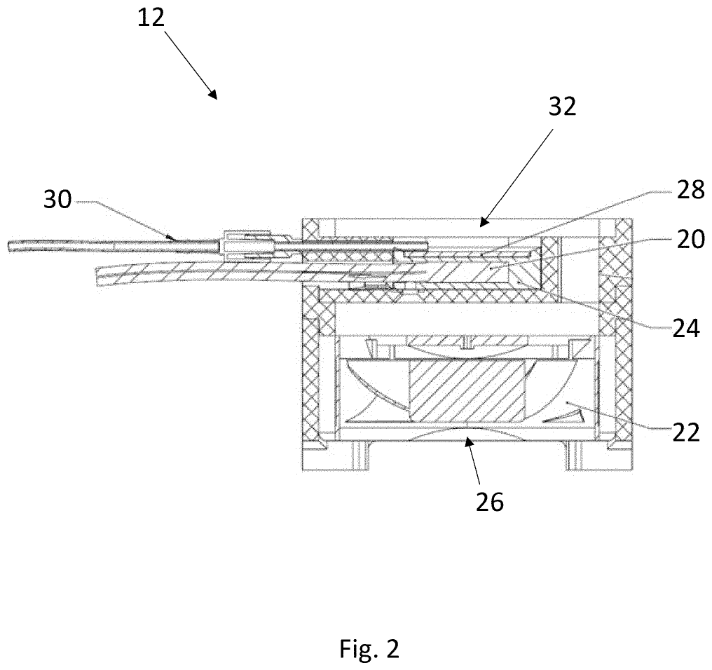

With reference to FIGS. 1 and 2, an exemplary, non-limiting sanitizing system 10 is shown that utilizes a dispensing device 12 and a filtering device 14. A sanitizing composition 16 is delivered to the dispensing device 12. As shown in FIG. 1, a pump 18 may deliver the sanitizing composition to the dispensing device 12. The dispensing device 12 includes a heater 20 and a fan 22 to evaporate the sanitizing composition. The fan 22 may be disposed below the heater 20. The dispensing device 12 includes an air inlet 26 for introducing air into the dispensing device. The heater 20 shown in FIG. 2 is configured as a cartridge heater that is within a heater block 24. An evaporative surface 28 is disposed adjacent to the heater 20. The evaporative surface 28 may be configured as a porous metal disc that is located in the heater block 24. The evaporative surface 28 may include a recess so that sanitizing composition does not run off the heater block and/or down to the fan. The evaporative surface 28 allows the sanitizing composition to wet out a large surface area, leading to faster and more efficient evaporation of the sanitizing composition. As shown in FIG. 1 for illustrative purposes only, the sanitizing composition may be delivered to the evaporative surface 28 by a tube 30 that is connected to pump 18, such as a micro pump. There are air flow paths around the heater block 24 that enable air from the fan 22 below to carry the air up and around the evaporative surface 28 and through an evaporation chamber 32. The dispensing device 12 and the filtering device 14 of FIG. 1 may be attached and configured as a single unit or may be separate components of the system 10 located in the same space.

As noted previously, the air flowing through the dispensing device 12 may be heated upstream and/or at the evaporative surface 28. The inventors have surprisingly found that doing so can greatly reduce the amount of liquid particles generated.

For pre-heating the air flowing through the dispensing device 12, a heater may be disposed adjacent the air inlet 26 either upstream or at the evaporative surface to raise the temperature of the air introduced or flowing through the device 12. Alternatively, or in conjunction with a pre-heater adjacent the air inlet 26, a second heater may be positioned adjacent the heater 20. Still in other configurations, the evaporative surface 28 and/or heater 20 may be configured to heat the air flowing through the dispensing device 12 adjacent and at the evaporative surface 28. Regardless of how heat is being provided, any suitable heater may be utilized.

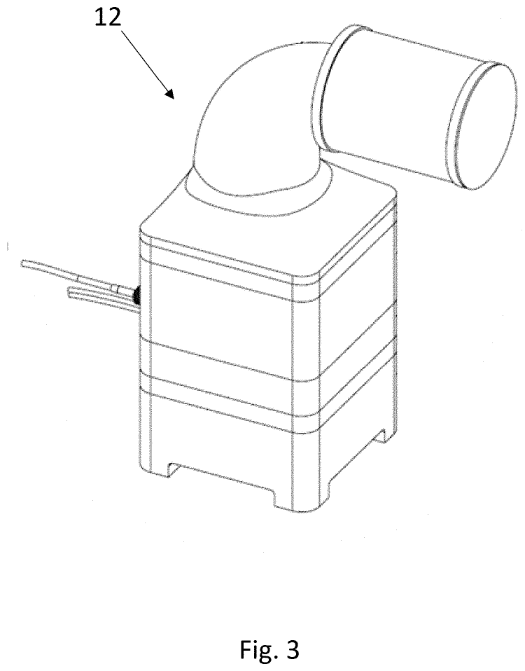

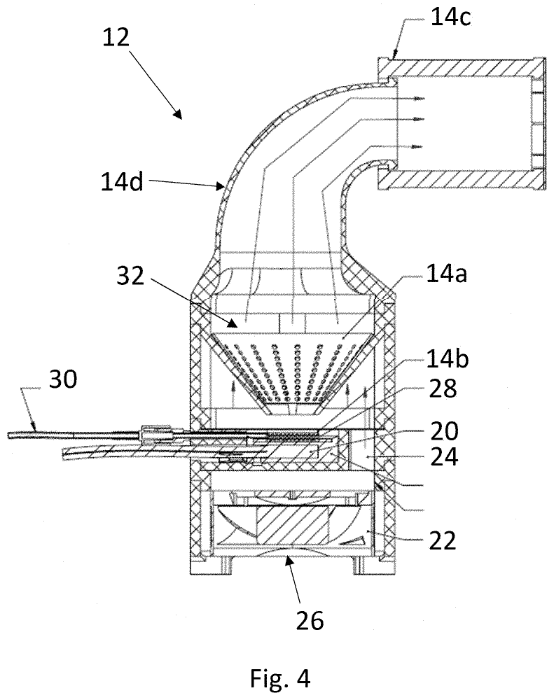

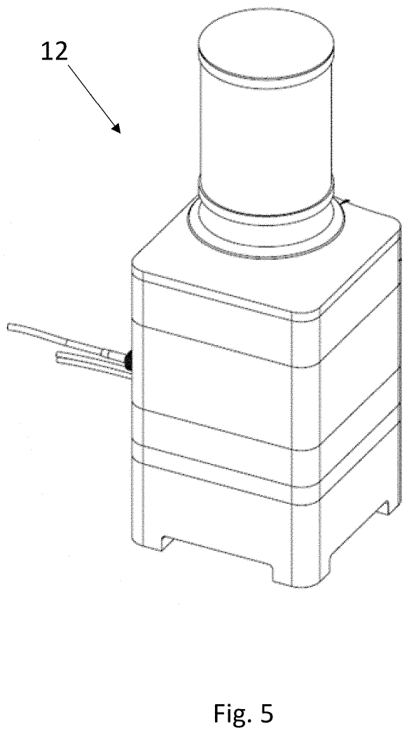

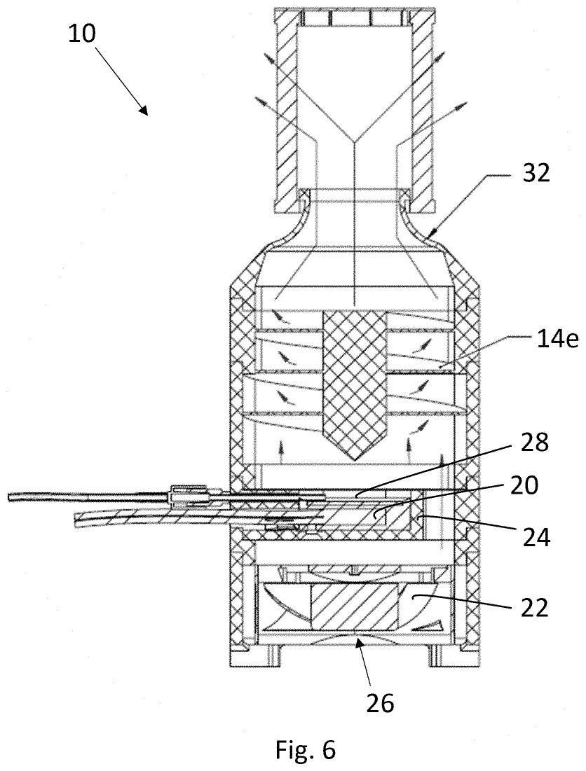

As discussed above, the dispensing device 12 may include a filtering device 14 to help remove liquid particles before they are dispensed into the air. As shown in FIGS. 3-6, within the evaporation chamber 32, a myriad of filtering devices may be utilized to help filter the liquid particles from the air. For example, liquid particles may be removed from the air by condensing them on surfaces and letting gravity lead the condensed sanitizing composition back to the evaporative surface. With reference to FIGS. 3-4, a conical shaped mesh 14a that lets air and vapor through to the evaporation chamber 32 but will trap some of the liquid particles, using gravity to direct the liquid particles back to the evaporative surface 28 may be utilized. As another example, with reference to FIG. 4, a metal mesh 14b or steel wool type construction over top of the evaporative surface to trap liquid particles may be utilized. As another example, with reference to FIGS. 5-6, a circular vortex element 14e that forces the air in a circular vortex pattern and causes larger liquid particles to move toward the outer surface of the evaporation chamber 32 where they are captured and flow back to evaporative surface 28. As another example, with reference to FIGS. 3-4, an elbow 14d, such as a 90-degree elbow, may be used to further collect liquid particles at the outer surface of the evaporation chamber 32 as the air flow and vapor make the 90-degree turn. The liquid particles collected on the outer surface can flow back to the evaporative surface 28. Lastly, a media filter 14c may also be attached at the outlet of the evaporation chamber 32 which can capture liquid particles.

It is worth noting that any suitable component or combinations of components may be utilized to reduce the amount of liquid particles being dispensed by the device 12. For example, any suitable combination of two or more of: the conical shaped mesh 14a, the vortex 14e, metal mesh 14b, 90-degree elbow 14d, and a media filter 14c, may be utilized. In such configurations, the filtering device 14 may comprise a first filter and a second filter wherein the first and second filters are discrete. Further, in such configurations, at least one of the first filter or second filter may be unitary with the dispensing device.

The dispensing device may have a delivery engine, such as a wick, that is used to transport a sanitizing composition and/or evaporate a sanitizing composition therefrom. The delivery engine may be configured in various ways. For example, the delivery engine may be in the form of a wick, membrane, gel, porous or semi-porous substrate, including a felt pad.

If the dispensing device includes a delivery engine in the form of a wick, the wick may be configured to have various different shapes and sizes. For example, the wick may have a cylindrical or an elongated cube shape. The wick may be defined by a length and a diameter or width, depending on the shape. The wick may have various lengths. For example, the length of the wick may be in the range of about 1 millimeter (“mm”) to about 100 mm, or from about 5 mm to about 75 mm, or from about 10 mm to about 50 mm. The wick may have various diameters or widths. For example, diameter or width of the wick may be at least 1 mm, or at least 2 mm, or at least 3 mm, or at least 4 mm. A wick may exhibit a density. The wick density may be in the range of about 0.100 grams/cm³ (“g/cc”) to about 1.0 g/cc. A wick may comprise a porous or semi-porous substrate. The wick may be composed of various materials and methods of construction, including, but not limited to, bundled fibers which are compressed and/or formed into various shapes via overwrap (such as a non-woven sheet over-wrap) or made of sintered plastics such as PE, HDPE or other polyolefins. For example, the wick may be made from a plastic material such as polyethylene or a polyethylene blend.

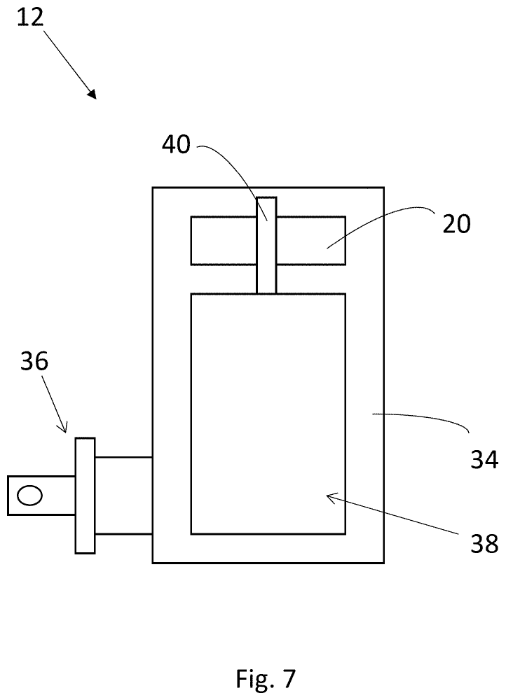

FIG. 7 illustrates an exemplary dispensing device 12 in the form of an electrical wall plug. The wall plug may include a housing 34, and the housing 34 is supported on an electrical outlet by a plug 36 that is at least indirectly joined to the housing 34. The dispensing device 12 further comprises at least one reservoir 38 for containing the sanitizing composition. The housing 34 may serve as a holder for the reservoir(s) and any of the other components of the dispensing device. The dispensing device comprises a delivery engine in the form of a wick 40 and a heater 20 for dispensing the volatile material. While FIG. 7 illustrates one reservoir, one heater, and one delivery engine, it is to be appreciated that the dispensing device may include more than one reservoir, heater, and/or delivery engine. If the dispensing device includes more than one reservoir, each reservoir may contain a different sanitizing composition or may contain the same sanitizing composition. A dispensing device such as shown in FIG. 7 may also utilize a fan to assist in evaporation. An exemplary dispensing device may utilize a cord or may be battery-operated instead of being plugged into the wall as shown in FIG. 7.

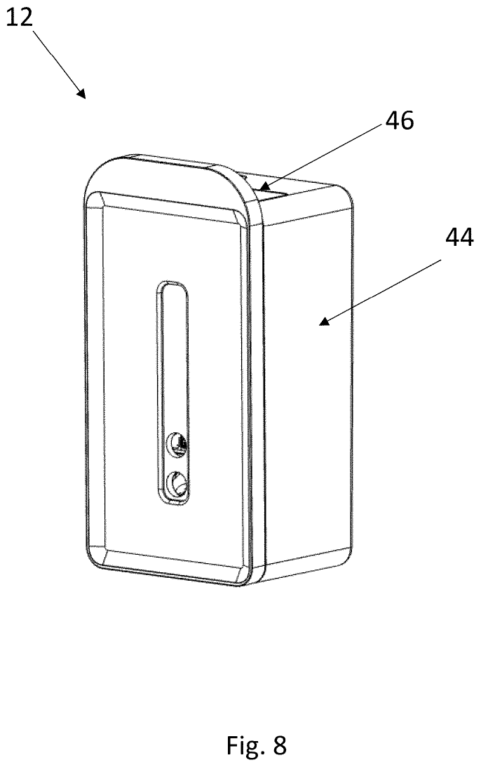

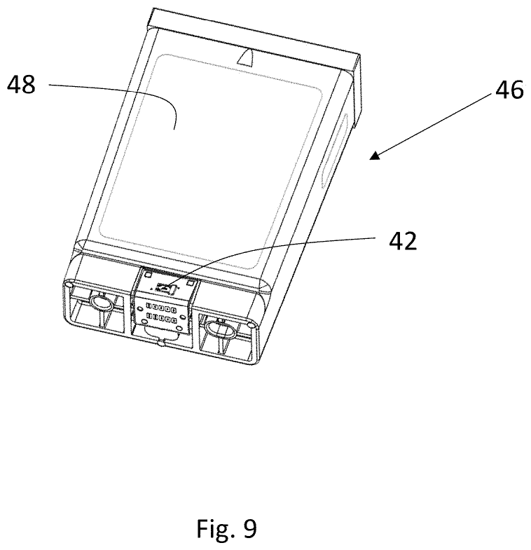

FIGS. 8 and 9 illustrate a dispensing device 12 that includes a housing 44 and a cartridge 46 removably connected with the housing 44 for dispensing the sanitizing composition. The cartridge 46 includes a reservoir 48 for containing the sanitizing composition and a microfluidic die 42. The microfluidic die 42 may include a heater(s) or piezo crystal(s) that is used to atomization the sanitizing composition to dispense the sanitizing composition into the atmosphere.

### EXAMPLES

Protocol:
1. Place hotplate set to 250° C. in a temperature & humidity-controlled room that is cubic meters in volume. The hotplate is positioned at the geometric center of the room. Also place TSI particle size analyzers (OPS 3330 and Nanoscan SMPS) and a RABBIT AIR™ HEPA filter in the room with the hotplate.
2. Set temperature of room to 21° C. and humidity to 50% relative humidity. Set total number of air exchanges in room to zero.
3. Set up particle size analyzers to measure number of liquid particles in the air per manufacturer's instructions. Input a refractive index value for TEG of 1.453.
4. Pipet 1.00 mL of TEG onto center of hotplate. This is time=0.
5. Set up impinger air samplers using 20 mL of acetonitrile as the solvent in each impinger. Air samplers are positioned at the greatest distance from the hotplate of step 1.
6. At time=7 min, collect a sample of air from the room by drawing air through the impinger at a rate of 50 mL/minute until a total volume of 100 mL of air has been drawn through the impinger.
7. Pour contents of each impinger into a clean glass vial with rubber septum screw cap.
8. At time=9 min, turn on the RABBIT AIR™ HEPA filter at the highest fan speed.
9. At time=24 min, turn off the RABBIT AIR™ HEPA filter.
10. At time=26 min, take another sample of the air using the impinger air samplers per the protocol in steps 6-7.
11. At time=30 min, stop the experiment and begin purge cycle of room.
12. Analyze the impinger samples via GC/MS analysis to discern total level of TEG in the air.

### Example 1: Evaluating Liquid Droplet Removal by HEPA Filtration

TABLE 1
Evaporation Method: TEG pipetted onto hotplate @ 250° C.

| | Total mass of 0.1-3 μm diameter | | | |
|---|---|---|---|---|
| | liquid particles in air (mg/m³) | | | |
| | | Filter | After 10 min | After 15 min |
| | Pre-Filter | powered on | of filtration | of filtration |
| | Time = | Time = | Time = | Time = |
| | 7 min | 11 min | 21 min | 26 min |
| Nil Filter | 1.36 | 0.83 | 0.25 | 0.17 |
| Nil Filter | 1.25 | 0.80 | 0.25 | 0.15 |
| Nil Filter | 1.17 | 0.68 | 0.22 | 0.16 |
| Nil Filter | 1.22 | 0.72 | 0.23 | 0.16 |
| With Filter | 1.17 | 0.48 | 0.02 | 0.005 |
| With Filter | 1.15 | 0.59 | 0.02 | 0.006 |
| With Filter | 1.68 | 0.71 | 0.02 | 0.006 |
| With Filter | 0.90 | 0.70 | 0.03 | 0.008 |
| With Filter | 1.34 | 1.05 | 0.03 | 0.008 |
| With Filter | 1.13 | 0.91 | 0.03 | 0.008 |

TABLE 2
Evaporation Method: TEG dosed from syringe
pump onto hotplate @ 33 mg/min

| | Total mass of 0.1-3 μm diameter | | | |
|---|---|---|---|---|
| | liquid particles in air (mg/m³) | | | |
| | Pre-Filter | After 10 | After 15 | After 20 |
| | Time = | min of | min of | min of |
| | 30 min | filtration | filtration | filtration |
| Nil Filter | 2.94 | 2.36 | 1.76 | 0.81 |
| With Filter | 3.47 | 0.25 | 0.02 | 0.003 |
| With Filter | 3.70 | 0.33 | 0.03 | 0.005 |

### Example 2: Evaluating Microorganism Reduction Pre and Post Filtration

TABLE 3
Total TEG, Total particles & Microorganism kill data
Evaporation Method: TEG pipetted onto hotplate @ 250° C.

| | Pre-Filtration | Post-Filtration |
|---|---|---|
| Total mass of 0.1-3 μm diameter | 1.12 | 0.008 |
| liquid particles in air (mg/m3) | | |
| Total TEG in air as measured | 34.42 | 3.85 |
| by GC/MS (mg/m3) | | |
| Microorganism kill against | 3 log10 reduction | 3 log10 reduction |
| Staphylococcus aureus | in 18.2 min | in 33 min |

* TEG level in the air after filtration is near the theoretical saturation level of TEG in the air (~4.42 mg/m3).

### Examples 3-5: Evaluating the Effect of Heat on the Level of Sanitizing Composition in the Air

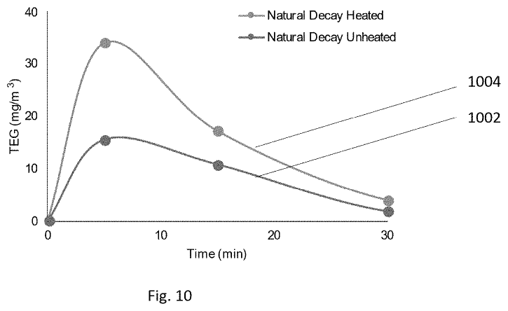

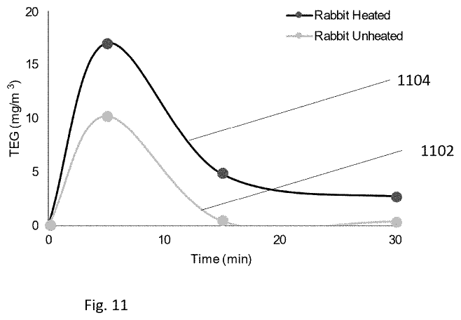

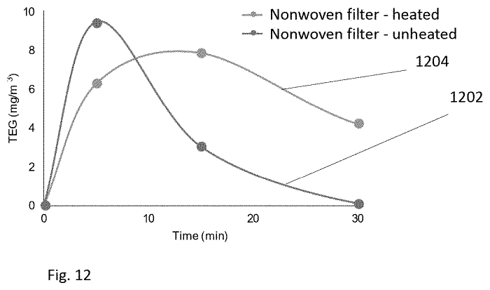

FIGS. 10-12 show data regarding Examples 3-5, respectively.
1. Place the exemplary, sanitizing system in accordance with the present disclosure in a temperature & humidity-controlled room that is 25 cubic meters in volume. The sanitizing system should be placed in the geometric center of the room.
2. Place the sanitizing system on top of the plastic L-shaped tube and connect a Steinel™ electronic heat gun HG 2310 LCD to the other end of the tube so that output air from heat gun travels through the tube.
3. Also place TSI particle size analyzers (OPS 3330 and Nanoscan SMPS) and a RABBIT AIR™ HEPA filter in the room with the sanitizing system.
4. Set temperature of room to 21° C. and humidity to 50% relative humidity. Set total number of air exchanges in room to zero.
5. Set up particle size analyzers to measure number of liquid particles in the air per manufacturer's instructions. Input a refractive index value for TEG of 1.453.
6. On the sanitizing system, set the setpoint temperature of evaporative surface to 230 C. Set fan speed to 100% output—50 cfm.
7. Turn on the heat gun. Set the temperature to 150 degrees F. and the fan speed to 7 by following the manufacturer's instructions.
8. Set up air samplers by placing a hydrophobic graphitized carbon absorbent resin tube in the inlet tube of each air sampling pump so that air in the room will pass through the resin for the specified amount of time.
9. Use the air sampler setup to collect a background air sample in the room. Sample air for 2 minutes at a rate of 2 L/min.
10. Once evaporative surface reaches 230 degrees C., use the peristaltic pump system on the sanitizing system to deliver 1.00 mL of triethylene glycol (TEG) to the recessed area of the evaporative surface at a rate of 1.00 mL per minute. The time at which TEG dispensing is started is considered time=0
11. At time=5 min, collect a sample of air from the room by drawing air through the hydrophobic graphitized carbon absorbent resin for a total of 2 minutes at a rate of 2 L air per minute.
12. At time=15 min, collect a sample of air from the room by drawing air through the hydrophobic graphitized carbon absorbent resin for a total of 2 minutes at a rate of 2 L air per minute.
13. At time=30 min, collect a sample of air from the room by drawing air through the hydrophobic graphitized carbon absorbent resin for a total of 2 minutes at a rate of 2 L air per minute.
14. At time=32 min, turn on the RABBIT AIR™ HEPA filter at the highest fan speed.
15. At time=42 min, turn off the RABBIT AIR™ HEPA filter.
16. At time=44 min, collect a sample of air from the room by drawing air through the hydrophobic graphitized carbon absorbent resin for a total of 2 minutes at a rate of 2 L air per minute.
17. Analyze the hydrophobic graphitized carbon absorbent resin samples via GC/MS analysis to discern total level of TEG in the air.
18. Repeat the above experiment according to steps 1-17, but do not use heat gun. Remove heat gun from the inlet of the L-shaped tube beneath the sanitizing system, so that only ambient temperature air (˜70 F) is drawn in by the fan.

TABLE 3
Total TEG in the air as measured by GC/MS (mg/m³)

| | Fan inlet | Fan inlet |
|---|---|---|
| | air at 70 F. | air at 150 F. |
| Before HEPA filtration | 15.54 | 34.05 |
| Time = 5 min | | |
| Before HEPA filtration | 10.88 | 17.29 |
| Time = 15 min | | |
| Before HEPA filtration | 1.96 | 4.09 |
| Time = 30 min | | |
| After 10 min of HEPA filtration | 1.75 | 2.62 |
| Time = 44 min | | |

TABLE 4
Data regarding Total TEG in the air with off-line filtration, continuous filtration, and in-line
filtration.

| | | | No In-Line Filtration | | | |
|---|---|---|---|---|---|---|
| | | | Off-line RABBIT AIR | | With In-Line | |
| | No In-line Filtration | | HEPA filter running | | Nonwoven filter | |
| | Off-line | | continuously during test | | Off-line | |
| | Heated | Unheated | | Unheated | | Unheated |
| Timepoint | Inlet Air | Inlet Air | Heated Inlet | Inlet Air | Heated Inlet | Inlet Air |
| (min) | (mg/m³) | (mg/m³) | Air (mg/m³) | (mg/m³) | Air (mg/m³) | (mg/m³) |
| 0 | 0.00 | 0.00 | 0.00 | 0.00 | 0.00 | 0 |
| 5 | 34.05 | 15.54 | 17.01 | 10.22 | 6.27 | 9.44 |
| 15 | 17.29 | 10.88 | 4.86 | 0.49 | 7.84 | 3.03 |
| 30 | 4.09 | 1.96 | 2.77 | 0.41 | 4.20 | 0.10 |
| 44 | 2.62 | 1.75 | 3.16 | 0.00 | 3.30 | 0.06 |

The nonwoven filter utilized for the in-line filtration was a multilayer 60 gsm nonwoven with two outer layers being 48 gsm collectively and the other layer being 12 gsm disposed therebetween. The 48 gsm layer included 50 percent by weight 1.3 denier trilobal polypropylene, 25 percent by weight 3.3 denier trilobal polypropylene and 25 percent by weight 1.7 denier viscose. The 12 gsm layer was a spunbond polypropylene layer sandwiched between the two outer layers. The filter material utilized is described in additional detail in U.S. Pat. No. 10,300,420B2.

Referring to FIGS. 10-12, as shown, the heated curves 1004, 1104, and 1204, respectively, allow for higher amounts of TEG to be present in the air for longer period of time if desired. In contrast, unheated curves 1002, 1102, and 1202, respectively show a much faster decay of TEG than their heated counterparts. The same holds true for when filtration is added to the sanitization system.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An air sanitizing system comprising:
a dispensing device configured to dispense a sanitizing composition into liquid particles and vapor;
a fan;
a filtering device configured to remove at least a portion of the liquid particles.

2. The system of claim 1, wherein the dispensing device and filtering device are separate components located in the same space.

3. The system of claim 1, wherein the dispensing device comprises a pump with an evaporative surface.

4. The system of claim 3, wherein the dispensing device comprises a heater disposed adjacent to the evaporative surface for evaporating the sanitizing composition.

5. The system of claim 1, wherein the dispensing device comprises the filtering device.

6. The system of claim 5, wherein the filtering device comprises at least one of a circular vortex, an elbow, a media filter, electrostatics, ionization, agglomeration.

7. The system of claim 1, wherein the filtering device comprises a media filter.

8. The system of claim 1, wherein the dispensing device comprises the filtering device which comprises a first filter, and wherein the filtering device comprises a second filter separate from the first filter.

9. The system of claim 8, wherein the first filter is comprised by the dispensing device and the second filter is discrete from the dispensing device.

10. The system of claim 1, wherein the sanitizing composition comprises a glycol ether.

11. The system of claim 10, wherein the glycol ether comprises from between about 50 wt. % to about 100 wt. %, based on the total weight of the sanitizing composition.

12. The system of claim 10, wherein the glycol ether comprises at least 75 wt. %.

13. The system of claim 1, further comprising a second heater for heating air flowing through at least a portion of the dispensing device.

14. The system of claim 1, wherein the fan has a capacity of from about 10 cubic feet per minute (cfm) to about 200 cfm, during evaporation of the sanitizing composition.

15. The system of claim 1, wherein the fan has a capacity of from about 22 cfm to about 100 during evaporation of the sanitizing composition.

16. The system of claim 1, wherein the fan has a capacity of from about 70 cfm to about 500 cfm during filtration of the liquid particles from the air.

17. The system of claim 1, wherein the fan has a capacity of from about 125 cfm to about 300 cfm during filtration of the liquid particles from the air.

18. The system of claim 1, further comprising a second fan, wherein the fan is utilized during evaporation of the sanitizing composition and the second fan is utilized during filtration of liquid particles from the air.

19. The system of claim 1, further comprising one or more of: a particle sensor, temperature sensor, humidity sensor, optical sensor, VOC sensor, occupancy sensor, and chemistry sensor.

20. The system of claim 19, wherein the one or more sensors are in signal communication, either directly or indirectly through a microprocessor, with at least one of the fan, dispensing device, evaporation device, or the filtering device.

21. The system of claim 19, further comprising a pump for delivering the sanitizing composition onto an evaporative surface and a heater for heating the evaporative surface, and wherein the one or more sensors are in signal communication, either directly or indirectly through a microprocessor, with at least one of the fan, the filtering device, the heater or the pump.

22. The system of claim 21, wherein the one or more sensors are utilized to control, at least in part, the concentration level of sanitizing composition in the air.
